SEQUENCE LISTING (28 sequences)

SEQ ID NO: 1 - DNA, 4161 bp, Mus musculus
  source 1..4161: mol_type = other DNA
gctttggcaaagccatccacacgtgacaaaacgtaaggaagtggaagaaaccgtctagagcaatatcaagtaccacttaattagagaatatttttttaaccttttcctcctcctgcgccgggtgtgtgatcccggagagcagagtccattcagcaccttggacagagccagcggatttgtccgaggtggtagtacttcatccaggtattcttttcctcgctgtcaagccaacccggtgtcgcccttaaaaagcgtcttttccgaggttcggctcacaccgagatcggggctggagagagagtcagattttggagcggagcgtttggagagcgagccccagtttggtcccctcattgagctcgctgaagttggcttcctagcggtgtaggctggaatagactcttggcaagctccgggttggtatactgggttaactttgggaaatgcaagtgtttatcaccaggatctagccaccggggtggtgtaagccgcaaagaagttccaccaggtgagaagagtgatgaccatccttttccttactatggttatttcatacttcggttgcatgaaggcggcgcccatgaaagaagtaaacgtccacggacaaggcaacttggcctacccaggtgtgcggacccatgggactctggagagcgtgaatgggcccagggcaggttcgagaggtctgacgacgacatcactggctgacacttttgagcacgtcatcgaagagctgctggatgaggaccagaaggttcggcccaacgaagaaaaccataaggacgcggacttgtacacttcccgggtgatgctcagcagtcaagtgcctttggagcctcctctactctttctgctggaggaatacaaaaattacctggatgccgcaaacatgtctatgagggttcggcgccactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacactgaccattaaaaggggaagatagtggatttatgttgtatagattatattgagacaaaattatctatttgtatatatacataacagggtaaattattcagttaagaaaaaataattttatgaactgcatgtataaatgaagtttatacagtacagtggttctacaatctatttattggacatatccatgacctgaaaggaaacagtcatttgcgcacaactttaaaagtctgcattacattcctcgataatgttgtggtttgttgccgttgccaagaattgaaaacaaaaagtttaaaaaaaataataataaattgcatgctgctttaattgtgaattgataataaactgtccctctttcagaaaacagattaaaaaaacaaaaaacaaaaaaaaaaaaacaaaaaacaaaaacaaaaattggaaccaaaacattccgtttacattttagacactaagtatcttcgttcttgttagtactctgttttactgctttcgacttctgatagcgttggaattaaaacaatgtcaaggtgctgttgtcattgctttactggcgtaagggacggggaatgggaggggtagatttctgtttgttttgtgttttattttgtttgtttgtttgttttgttttttagttccacccggagtagggatggagaaaatttcttcactatccattctggttgataaagcgttacatttgtatgttgtaaagatgtttgcaaaatccaatcagatgactggaaaacaaataaaaattaaggcaactgaataaaatgctcacactccactgcccatgatgtatctccctggtccccctcagctcactcttctggcatgggtcagggaaaattgcttttattggaaagaccagcatttgttcaaagcatactctttccctccctcctcccattttggtcccttctttttgttttgttttaagaaagaaaattaagttgcgcgctttaaaatattttactactgctacaaacagatgaacaatgtgtgtcattttatgacactcatggaaaacagtgatttttttttaccctaaagaaaaacaaataaaaataacccaaaatattctttttttaaaaggcataaatattgggtaaattgtaatatggcctaacagtgtttgcagataaaagttattgtatacacccagatacttagataagagcagggatccacactgccattgaaataggactgaatggccctgcggaggctaagtggagctgacatactatttcctggcagtgcaggaggaatttctgagtggccatcctaaggtctaggatggaggtggggaatggtacttgagacattcctaaaggaaggctcggaagcacccttcagagcaggctctggaatgatgtgtcaagttgcttaggccttctgctttaagtgcctacattacctaacagtgctcaagaggttctcgattggagaaccacactcaaatccatttatagcctccatcccatttctaaataattgtgtataaagttggattaacctggagcaactttggatccaaatatggcacagcaataatgatattaatgcagcatgatgggaaatgtttgctgtgaagagaattgatttgctttgagcttagacttcaggaagcctaggttttttatttttttatttttgagacattttggtaaaaggaaaaaaagaaaacaaacaaacaaacaaacaaaaccagaaaaagcatcaaaactcaggcagaatgagcaatgtctgaaagggctagaaaaacaagacatagcaaggtgctttcactgtgaaagagacaagaacacaggaggaaatattgcttcagtgaagagcacagacggctcctgccaatttattacaagagtcccgtctgtactttaccctttggggttagaagtcaagttggaagcctgaatgaatggacccaatgagaactagtgttaagcccatttccctagtcaggtttttttcaagcgtgaatgtgttagtggttactctcctgggttcctgagcatcagaaaaaaaaaaaaaaagaggcaaacaatcgcttcatcttaggagtggaaaggaaacagaagtggacgtccgctgtgactcagggagtgaagataccatcagcaaatagtttcttttttgttcattcgttcctttcgagttagcctgtcttttggaataccactgaatatgctgtttttgaaagacttcatgtagcatagattgttttgtgccgtttaccaaattaacctttgtcatcgttttttaacctattcaggaatgcttggaatatctgctctatgttaactttttgcagcttcattctgagagacattagtcaaacaaacaaaaggatccccatcacaatcttacagtactgcaagggccaggtctgttaatcggcttcacaggagacatcagcaattgtgtggtcagtggctggctctcttacccactaagatacatcatagctacatgttggtggtttatgttgacctgagatttatttgttaaaatctcttcttcgtttctgttcgttctggttctgttctgttctgttctgttctgttttggttttaaagtcttgctgtggtctcttgttggcagaaatgttttatgcatggcagcaggcctgttgcttttttatagtgattcccattgaaactgtaagtaaatgtctgtggccttgttctctctatggtaaagatattattcaccatgtaaaacaagaaaaaatatttattgtattttagtatatttatataattatgttattgaaaaaattggcattaaaacttaaccacatcagaagcctattgtaaatacaggttctatttaagtgtaccaattaacatataatatatgttttaaatatagaatttttaatgtttttaaatatattttcaaagt

SEQ ID NO: 2 - DNA, 750 bp, Mus musculus
  source 1..750: mol_type = other DNA
atgaccatccttttccttactatggttatttcatacttcggttgcatgaaggcggcgcccatgaaagaagtaaacgtccacggacaaggcaacttggcctacccaggtgtgcggacccatgggactctggagagcgtgaatgggcccagggcaggttcgagaggtctgacgacgacatcactggctgacacttttgagcacgtcatcgaagagctgctggatgaggaccagaaggttcggcccaacgaagaaaaccataaggacgcggacttgtacacttcccgggtgatgctcagcagtcaagtgcctttggagcctcctctactctttctgctggaggaatacaaaaattacctggatgccgcaaacatgtctatgagggttcggcgccactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacactgaccattaaaaggggaagatag

SEQ ID NO: 3 - protein, 249 aa, Mus musculus
MTILFLTMVISYFGCMKAAPMKEVNVHGQGNLAYPGVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 4 - DNA, 4169 bp, Rattus norvegicus
  source 1..4169: mol_type = other DNA
gctttggcaaagccatccgcacgtgacaaaacgtaaggaagtggaagaaaccgtctagagcaatatcaagtaccacttaattagagaatatttttttaaccttttcctcctgctgcgccgggtgtgtgatccgggcgagcagagtccattcagcaccttggacagagccagcggatttgtccgaggtggtagtacttcatccaggtattcttttcctcgctgtcaagccaacccggtgtcgcccttaaaaagcgtcttttccgaggttcggctcacactgagatcggggctggagagagagtcagattttggagcggagcgtttggagagccagccccagtttggtcccctcattgagctcgctgaagttggcttcctagcggtgtaggctggaatagactcttggcaagctccgggttggtatactgggttaactttgggaaatgcaagtgtttatctccaggatctagccaccggggtggtgtaagccgcaaagaagttccaccaggtgagaagagtgatgaccatccttttccttactatggttatttcatacttcggttgcatgaaggctgcgcccatgaaagaagcaaacgtccacggacaaggcaacttggcctacccagctgtgcggacccatgggactctggagagcgtgaatgggcccagggcaggttcgagaggtctgacgacgacgtccctggctgacacttttgagcacgtgatcgaagagctgctggatgaggaccagaaggttcggcccaacgaagaaaaccataaggacgcggacttgtacacttcccgggtgatgctcagcagtcaagtgcctttggagcctcctctgctctttctgctggaggaatacaaaaattacctggatgccgcaaacatgtctatgagggttcggcgccactccgaccccgcccgccgtggggagctgagcgtgtgtgacagtattagcgagtgggtcacagcggcagataaaaagactgcagtggacatgtccggtgggacggtcacagtcctggagaaagtcccggtatcaaaaggccaactgaagcaatatttctacgagaccaagtgtaatcccatgggttacacgaaggaaggctgcaggggcatagacaaaaggcactggaactcgcaatgccgaactacccaatcgtatgttcgggcccttactatggatagcaaaaagagaattggctggcggttcataaggatagacacttcctgtgtatgtacactgaccattaaaaggggaagatagtggatttatgttgtatagattatattgagacaaaaattatctatttgtatatatacataacagggtaaattattcagttaagaaaaagataattttatgaactgcatgtataaatgaagtttatacagtacagtggttctacaatctatttattggacatatccatgaccagaaagaaacagtcatttgcgcacaactttaaaagtctgcattacattcctcgataatgttgtggtttgttgccgttgccaagaattgaaaacaaaaagttaaaaaaaataataaattgcatgctgctttaattgtgaattgataataaactgtccctctttcagaaaacagacaaaaaaacaaaaaacaaaaaaaagcaaaaacaaaaatttgaaccaaaacattccgtttacattttagacactaagtatcttcgttcttgttagtactctgttctactgctttcaacttctcatagcgttggaattaaaactatgtcaaggtgctgttgtcattgctttactggcttaggggatggggaacgggaggggtagatttctgtttgttttgtgttttatttcgtttgtttgtttgttttgtttttttagttccacccggagtagggatggagaaaatttcttcactctccattctggttgataaagcgttacatttgtatgttgtaaaaaatgtttgcaaaatccaatcagatgactggaaaacgaataaaaattaaggcaactgaataaaatgctcacacaacactgcccatgatgtatctccctggtcccccaggtcactcttctggcatgggtcagggaaagctgcttttattggaaagaccagcatttgtttaaagcacattctttccctccctcctcccattttggtccctttcttttttgttttgttttaagaaagaaaattaagttgcgcgctttgaaatattttatcactgctgtgaacagatgaacaatgtgtgtcatttcatgacactcgtggaaaacagtgattttttttttattttttgccctaaggagaaacaagtaagaataaccgaaaatgttctttttttttaaaggcataaacagtggataagttataatatggcctaacaatgtttgcagataaaagatattgcatacagccagatactagagcagggatccacactgccactgaaatgcgactgaatggccctgtggaggctaagtggagctgacatactatttcctggcagagcaggaggaatttctgagtggccatcctgaggtctagatggaggtggggaatggtacttgagacattcctaaaggaaggctcggaagcacccttcagagcaggctctggaatgatgtgtcaagtttcttaggccttctgctttaagtgcctacgttacctaacagtgctcaagaggttctcaattggagaaccacactcaaatccatttatggcctccatcccattttaaataattatggataaagttggattaacctggagcagctttggatccaaatatggcatagcagtgatgctatcagtgcagcatgatgggaaatgtttgctgtgaagagacttaactttctttgcgcttagacttcaggaagcctaggttttatttatttatttttttgagacattttggtgaaaggaaaaaagaaagaagaaaacaaacaaacaaaaccagaaaaagcaccaaaacttaggcagaatgagcaatgtctgtctgtaagggctagaatgacaaggcataggaaggtgctttcactgtgaaagagacaagaacacaggaggaaatactgcttaagtgaagagcacagaaagctcctgatagttctgtccattcagcacaagggtcccttctacactttacctcttggggttaggagaagtcaagctggaagcctgaatgaatggccccaatgagaactagtgttaagcccatttccctagtgaggttttccgccagcgcgaatgtgttagtggttacctgactgggctcctgggcatcagaaaaagaggcaaacaattgcttcatcttaggagtggaaagggtgaaacaaagtggctgtccactgtgactcagggagtgaagataccatcagcaaatagtttctttttgttcaactgttcctttagaactagtctgtcttctggagtcccactgaatcccctgtttttggaagacttcacgtagcctagattgttttgtgccgtttgacaacattaatctctgtcatcatttttaacctattaaggaatgctttgaatatctgctatatgctaactttttgcagcttcattctgagagacgttagtcaaacaaataaaaggagccccatcacaatctcacggtattcgaagggccaggtcgattaggtggcttcataggagaccctccgcaactgtgtggtcagtggctggctctcatacccactaagatacatcatagctccatgtcggtggtttatgttgacctgagattgatttgttaaaatctctcctctgtttctgttcgttctgtttccgtcctgttctgttctgttctgttctgaaagtcttgctgtggtctctttttggcagaagtgtttcatgcatggcagcaggcctgatgctttttatagtgattcccattgaaactgtaagtaaatgtctgtggccttgttctctctatggtaaagatattattcaccatgtaaaacaagaaaaatatttattgtattttagtatatttatataattatgttattgaaaaaaattggcattaaaacttaaccacatcagaagcctattgtaaatacaggttctatttaagtgtaccaattaacatataatatatgttttaaatatagaatttttaatgtttttaaatatattttcaaagtacataaaaaaaaaaaaaaaa

SEQ ID NO: 5 - DNA, 750 bp, Rattus norvegicus
  source 1..750: mol_type = other DNA
atgaccatccttttccttactatggttatttcatacttcggttgcatgaaggctgcgcccatgaaagaagcaaacgtccacggacaaggcaacttggcctacccagctgtgcggacccatgggactctggagagcgtgaatgggcccagggcaggttcgagaggtctgacgacgacgtccctggctgacacttttgagcacgtgatcgaagagctgctggatgaggaccagaaggttcggcccaacgaagaaaaccataaggacgcggacttgtacacttcccgggtgatgctcagcagtcaagtgcctttggagcctcctctgctctttctgctggaggaatacaaaaattacctggatgccgcaaacatgtctatgagggttcggcgccactccgaccccgcccgccgtggggagctgagcgtgtgtgacagtattagcgagtgggtcacagcggcagataaaaagactgcagtggacatgtccggtgggacggtcacagtcctggagaaagtcccggtatcaaaaggccaactgaagcaatatttctacgagaccaagtgtaatcccatgggttacacgaaggaaggctgcaggggcatagacaaaaggcactggaactcgcaatgccgaactacccaatcgtatgttcgggcccttactatggatagcaaaaagagaattggctggcggttcataaggatagacacttcctgtgtatgtacactgaccattaaaaggggaagatag

SEQ ID NO: 6 - protein, 249 aa, Rattus norvegicus
MTILFLTMVISYFGCMKAAPMKEANVHGQGNLAYPAVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 7 - DNA, 2196 bp, synthetic construct
  source 1..2196: mol_type = other DNA
  misc_feature 1..850: note = 5' homology arm
  misc_feature 805: note = silent PAM mutation
  misc_feature 817: note = silent PAM mutation
  misc_feature 851..871: note = spacer
  misc_feature 872..1387: note = NLuc
  misc_feature 1388..1393: note = spacer
  misc_feature 1394..2196: note = 3' homology arm
ttacaagcagatgggccacatgctgtccccgagaaagaaagttctaacctgttctgtgtctgtctctgcttccttcccacagttccaccaggtgagaagagtgatgaccatccttttccttactatggttatttcatacttcggttgcatgaaggcggcgcccatgaaagaagtaaacgtccacggacaaggcaacttggcctacccaggtgtgcggacccatgggactctggagagcgtgaatgggcccagggcaggttcgagaggtctgacgacgacatcactggctgacacttttgagcacgtcatcgaagagctgctggatgaggaccagaaggttcggcccaacgaagaaaaccataaggacgcggacttgtacacttcccgggtgatgctcagcagtcaagtgcctttggagcctcctctactctttctgctggaggaatacaaaaattacctggatgccgcaaacatgtctatgagggttcggcgccactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacacaatcgtatgttcgagcccttactatggatagcaaaaagagaattggctggcgattcataagaatagacacttcatgtgtatgtacactgaccattaaaaggggaagacgggatccaccggtcgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaagatcttagtggatttatgttgtatagattatattgagacaaaattatctatttgtatatatacataacagggtaaattattcagttaagaaaaaataattttatgaactgcatgtataaatgaagtttatacagtacagtggttctacaatctatttattggacatatccatgacctgaaaggaaacagtcatttgcgcacaactttaaaagtctgcattacattcctcgataatgttgtggtttgttgccgttgccaagaattgaaaacaaaaagtttaaaaaaaataataataaattgcatgctgctttaattgtgaattgataataaactgtccctctttcagaaaacagattaaaaaaacaaaaaacaaaaaaaaaaaaacaaaaaacaaaaacaaaaattggaaccaaaacattccgtttacattttagacactaagtatcttcgttcttgttagtactctgttttactgctttcgacttctgatagcgttggaattaaaacaatgtcaaggtgctgttgtcattgctttactggcgtaagggacggggaatgggaggggtagatttctgtttgttttgtgttttattttgtttgtttgtttgttttgttttttagttccacccggagtagggatggagaaaatttcttcactatccattctggttgataaagcgttacatttgtatgttgtaaagatgtttgcaaaatccaatcagatgactggaaaacaaataaaaattaaggcaactgaataaaatgctcacactccactgcccatgatgtatctccctggtccccctca

SEQ ID NO: 8 - protein, 427 aa, synthetic construct
MTILFLTMVISYFGCMKAAPMKEVNVHGQGNLAYPGVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGRRDPPVATMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 9 - DNA, 516 bp, synthetic construct
  source 1..516: mol_type = other DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 10 - DNA, 21 bp, synthetic construct
  source 1..21: mol_type = other DNA
cgggatccaccggtcgccacc

SEQ ID NO: 11 - skipped

SEQ ID NO: 12 - DNA, 23 bp, synthetic construct
  source 1..23: mol_type = other DNA
cctgtgtatgtacactgaccatt

SEQ ID NO: 13 - DNA, 23 bp, synthetic construct
  source 1..23: mol_type = other DNA
gaattggctggcgattcataagg

SEQ ID NO: 14 - DNA, 26 bp, synthetic construct
  source 1..26: mol_type = other DNA
acttgcttagaaaccgtttactctca

SEQ ID NO: 15 - DNA, 34 bp, synthetic construct
  source 1..34: mol_type = other DNA
aaatccactaagatctttacgccagaatgcgttc

SEQ ID NO: 16 - DNA, 20 bp, synthetic construct
  source 1..20: mol_type = other DNA
gtacatatcggccaccaaag

SEQ ID NO: 17 - DNA, 19 bp, synthetic construct
  source 1..19: mol_type = other DNA
tgccttttgtctatgcccc

SEQ ID NO: 18 - DNA, 20 bp, synthetic construct
  source 1..20: mol_type = other DNA
aggacaatcctttggatcgg

SEQ ID NO: 19 - DNA, 23 bp, synthetic construct
  source 1..23: mol_type = other DNA
aaactgattgctgaaaatggtgt

SEQ ID NO: 20 - DNA, 24 bp, synthetic construct
  source 1..24: mol_type = other DNA
aaatcttcgagtgtgaagaccatt

SEQ ID NO: 21 - DNA, 31 bp, synthetic construct
  source 1..31: mol_type = other DNA
tctgcgaattcatgaccatccttttccttac

SEQ ID NO: 22 - DNA, 31 bp, synthetic construct
  source 1..31: mol_type = other DNA
ttgatctcgagctatcttccccttttaatgg

SEQ ID NO: 23 - DNA, 30 bp, synthetic construct
  source 1..30: mol_type = other DNA
ctctctcgagtaaagcagtagccggctggt

SEQ ID NO: 24 - DNA, 30 bp, synthetic construct
  source 1..30: mol_type = other DNA
ctctgaattctgtggctttgctgtcctgga

SEQ ID NO: 25 - DNA, 30 bp, synthetic construct
  source 1..30: mol_type = other DNA
ctctctcgaggctttggcaaagccatccac

SEQ ID NO: 26 - DNA, 30 bp, synthetic construct
  source 1..30: mol_type = other DNA
ctctgaattccactcttctcacctggtgga

SEQ ID NO: 27 - DNA, 30 bp, synthetic construct
  source 1..30: mol_type = other DNA
ctctctcgagacccactttcccattcaccg

SEQ ID NO: 28 - DNA, 30 bp, synthetic construct
  source 1..30: mol_type = other DNA
ctctctcgagccaatcgaagctcaaccgaa

(19) United States
(12) Patent Application Publication
(10) Pub. No.: US 2026/0231914 A1
(43) Pub. Date: Aug. 13, 2026

(54) BRAIN-DERIVED NEUROTROPHIC FACTOR-NANO LUCIFERASE TRANSGENIC RODENTS AND METHODS OF USE THEREOF

(71) Applicant: University of Florida Research Foundation, Incorporated, Gainesville, FL (US)

(72) Inventors: Guey-Ying LIAO, Gainesville, FL (US); Baoji XU, Gainesville, FL (US)

(21) Appl. No.: 19/152,625

(22) PCT Filed: Jan. 31, 2024
(86) PCT No.: PCT/US2024/013774

§ 371 Date: Jul. 31, 2025

Related U.S. Application Data
(60) Provisional application No. 63/482,927, filed on Feb. 2, 2023.

(51) Int. Cl. A01K 67/0278; C07K 14/475; C12N 5/00; C12N 5/0793; C12N 9/22; C12N 15/11; C12N 15/85; C12N 15/90; G01N 33/50

(52) U.S. Cl. CPC A01K 67/0278; C07K 14/475; C12N 5/0018; C12N 5/0619; C12N 9/226; C12N 15/111; C12N 15/8509; C12N 15/907; G01N 33/5023; G01N 33/5058; A01K 2217/072; A01K 2227/105; A01K 2267/0393; C07K 2319/60; C12N 2015/8527; C12N 2310/20; C12N 2510/00

(57) ABSTRACT

Described are transgenic rodents that express a brain-derived neurotrophic factor-nano luciferase fusion protein (BD-NF-NLuc) and methods of making the BDNF-NLuc rodents. Also described are methods involving these rodents and/or cell populations derived from these rodents to screen BDNF-modulating molecules.

### CROSS-REFERENCE TO RELATED APPLICATIONS

This application claims the benefit of U.S. Application No. 63/482,927, filed Feb. 2, 2023, which is herein incorporated by reference, in its entirety, for all purposes.

### STATEMENT REGARDING FEDERALLY SPONSERED RESEARCH OR DEVELOPMENT

This invention was made with government support under Grant Nos. R01 DK105954 and R01 DK103335, awarded by the National Institutes of Health. The government has certain rights in the invention.

### REFERENCE TO A SEQUENCE LISTING SUBMITTED AS AN XML FILE VIA EFS WEB

The Sequence Listing written in file 607651SEQLIST.xml is 40 kilobytes, was created on Jan. 17, 2024, and is hereby incorporated by reference.

### BACKGROUND

Brain-derived neurotrophic factor (BDNF), a 28-kD dimeric secreted growth factor, binds to TrkB receptor tyrosine kinase, inducing its dimerization and activation. This activates many signaling cascades that cooperatively promote neuronal survival, regulates the development of neural circuits, stimulates synapse formation, enhances synaptic transmission, and facilitates synaptic plasticity in many brain regions. Deficiencies in BDNF have been linked to several brain disorders, making compounds that can alter neuronal BDNF attractive as potential therapeutics. However, sensitive and quantitative BDNF assays, including high-throughput screening (HTS) assays, are still needed.

### SUMMARY OF THE CLAIMED INVENTION

The invention provides a rodent comprising a genetically modified endogenous Bdnf locus, wherein the genetically modified endogenous Bdnf locus encodes a BDNF-reporter fusion protein, wherein a nucleic acid sequence encoding the reporter is inserted in frame with the endogenous Bdnf coding sequence and within 1, 2, 3, 4, 5, 6, 7, 8, 9, or 10 codons of the stop codon of the Bdnf gene.

The invention further provides that the reporter comprises an enzyme or a fluorescent protein, and optionally, the reporter comprises nano luciferase (NLuc). The invention provides that the expression of the Bdfn-NLuc mRNA and/or the BDNF-NLuc fusion protein approximate the tissue distribution and physiological functions of wild type Bdnf mRNA and BDNF.

The invention provides that the rodent is heterozygous for the genetically modified endogenous Bdnf locus. Optionally, the rodent is homozygous for the genetically modified endogenous Bdnf locus. The invention also provides that the rodent is a rat or a mouse; optionally a mouse.

The invention provides that the coding sequence of the genetically modified endogenous Bdnf locus encodes a BDNF-NLuc fusion protein comprising the sequence set forth in SEQ ID NO: 8.

The invention further provides a method of making the rodent described above, comprising: (a) introducing into a rodent one-cell stage embryo: (i) an donor template comprising an insert nucleic acid flanked by a 5′ homology arm that hybridizes to a 5′ target sequence at the endogenous Bdnf locus and a 3′ homology arm that hybridizes to a 3′ target sequence at the endogenous Bdnf locus, wherein the insert nucleic acid comprises the nucleic acid sequence encoding the reporter; and (ii) a nuclease agent targeting a target sequence within the endogenous Bdnf locus, wherein the genome is modified to comprise the genetically modified endogenous Bdnf locus; and (b) implanting the modified rodent one-cell stage embryo into a surrogate mother to produce a genetically modified F0 generation rodent comprising the genetically modified endogenous Bdnf locus.

The invention provides that the nuclease agent in step (a) (ii) is a Cas9 protein and a guide RNA that targets a guide RNA target sequence within the endogenous Bdnf locus. Optionally, step (a) further comprises introducing into the rodent one-cell stage embryo a second guide RNA that targets a second guide RNA target sequence within the endogenous Bdnf locus.

The invention also provides a method of assessing the activity of a molecule in modulating Bdnf expression, activity, and/or stability, comprising: (a) isolating neuronal cells from the brains of embryos of the rodent described above; (b) contacting the isolated neuronal cells with the molecule in vitro; and (c) assessing expression, activity, and/or stability of the reporter. Optionally, the neuronal cells comprise cortical and/or hippocampal neurons. Optionally, the neuronal cells are isolated from about embryonic day 15.5 (E15.5) to about embryonic day 20.5 (E20.5) embryos. Optionally, the neuronal cells are isolated at about embryonic day 18.5 (E18.5)

The invention further provides that the method of assessing the activity of a molecule in modulating Bdnf optionally comprises: (i) culturing the neuronal cells in Neurobasal medium containing about 2% fetal bovine serum (FBS), about 2% B27, about 2 mM GlutaMAX, about 50 U/mL penicillin/streptomycin (pen/strep), and about 16 μM 5-fluoro-2′-deoxyuridine (FUdR) for 3 to 12 days in vitro (DIV3-DIV12) at 37° C. and 5% CO₂ prior to step (c); and/or (ii) plating the neuronal cells at a density of about 1.0×10⁴ cells per well in a 384-well plate or about 4.5×10⁴ cells per well in a 96-well plate. Optionally, the neuronal cells are cultured for about 7 days in vitro (DIV7) prior to step (c).

The invention further provides that in the method described above, the reporter is Nluc, and the assessing involves assessing expression, activity, and/or stability of luciferase activity in the neuronal cell population. Optionally, one or more of the steps in the method described above are performed by an automated liquid handling system.

The invention provides a neuronal cell population comprising a plurality of neuronal cells derived from the from the brains of embryos of the rodent described above.

The invention also provides a method of assessing the activity of a BDNF-modulating molecule, comprising: (a) administering the BDNF-modulating molecule to the rodent described above; and (b) assessing the activity of the reporter in the rodent. Optionally, the reporter comprises NLuc and assessing the activity of the reporter in the rodent comprises bioluminescent imaging of the rodent, tissues from the rodent, or tissue extracts from the rodent.

### BRIEF DESCRIPTION OF THE FIGURES

FIGS. 1A-1E show the generation of Bdnf^{NLuc/+} mice. (FIG. 1A) Two sgRNAs (sgRNA1: CCTGTGTATGTACACTGACCATT [SEQ ID NO: 12] and sgRNA2: GAATTGGCTGGCGATTCATAAGG [SEQ ID NO: 13]) were used to increase knock-in efficiency. The donor plasmid was designed such that the nano luciferase (NLuc) coding sequence would be inserted immediately before the Bdnf stop codon. Red asterisks indicate the location of the stop codon. The two arrows denote the locations of two PCR primers used in (B). (FIG. 1B) Detection of the Bdnf^{NLuc} allele with genomic DNA PCR, using a forward primer outside the homology arm in the donor plasmid (PCR primer 1; SEQ ID NO: 14) and a reverse primer complementary to the NLuc coding region (PCR primer 2; SEQ ID NO: 15). The PCR generated a 1.5-kb product from the Bdnf^{NLuc} allele. (FIG. 1C) RT-PCR detection of Bdnf mRNA variants in the cortex of WT and BdnfNLuc/+ mice. The forward primer was complementary to Bdnf exon 4 (PCR primer 3; SEQ ID NO: 16), and the reverse primer was complementary to either the Bdnf coding sequence for detecting exon 4 Bdnf mRNA variant (0.86 kb PCR product [PCR primer 4; SEQ ID NO: 17]) or the NLuc coding sequence for detecting exon 4 Bdnf-NLuc mRNA variant (1.16 kb PCR product [PCR primer 5; SEQ ID NO: 18]). (FIG. 1D) RT-PCR detection of Bdnf mRNA or Bdnf-NLuc mRNA in WT and Bdnf^{NLuc/+} brain tissues. The forward primer was complementary to Bdnf exon 9 (PCR primer 6; SEQ ID NO: 19), and the reverse primer was complementary to the NLuc coding sequence (PCR primer 7; SEQ ID NO: 20). (FIG. 1E) NLuc activities in cortical and striatal lysates. Unpaired t test, ****p<0.0001; n=3 female mice. Error bars represent SEM.

FIGS. 2A-2C show culturing of cortical neurons from newborn Bdnf^{NLuc+} mice. (FIG. 2A) Activity of NLuc in DIV11 Bdnf^{NLuc/+} neurons cultured in a 96-well plate. Unpaired t test, ****p<0.0001; n=5-6 wells. (FIG. 2B) Differential interference contrast image, after 11 days in vitro (DIV11) Bdnf^{NLuc/+} neurons cultured in a 384-well plate. (FIG. 2C) NLuc activity in DIV11 Bdnf^{NLuc/+} neurons cultured in a 384-well plate. NLuc activities in cells and media were measured separately (n=56 wells). Error bars represent SEM.

FIGS. 3A-3E show culturing of embryonic Bdnf^{NLuc/+} neurons in 384-well plates. (FIG. 3A) NLuc activity in media at different ages of neuronal cultures. Each well contained 80 μl of medium, 20 μl of which was used for measurement of NLuc activity. Each cluster of values represents NLuc activity from one row of wells, except the two edge wells. CV for each row is shown above each cluster of value points. (FIGS. 3B-3E, respectively) Immunocytochemistry with antibodies against NeuN (neuronal marker), GFAP (astrocytic marker), MAP2 (dendritic marker), and PSD95 (postsynaptic marker) was performed on cultured cells at DIV7. Scale bars, 50 μm.

FIGS. 4A-4D show KCl and deltamethrin (DM) stimulation of BDNF synthesis in cultured Bdnf^{NLuc/+} neurons. Chemicals were added on DIV7, and NLuc activities were measured on DIV8. (FIG. 4A) KCl at 10 mM increased levels of BDNF-NLuc in both neurons and media. The two tests were done in two different cultures. n=6 wells/condition. Z′ is calculated using the following formula: 1−(3σₚ+3σₙ)/(|μₚ−μₙ|) where σ, μ, p, and n stand for standard deviation, mean, positive control (KCl) and negative control (vehicle), respectively. Unpaired t test: p<0.0001. DM at 2-10 μM increased levels of BDNF-NLuc in both neurons (FIG. 4B) and media (FIG. 4C). The numbers above columns are Z factors. n=6 wells/condition. One-way ANOVA with Dunnett's multiple comparison test vs. vehicle: ***p<0.001 and ****p<0.0001. (FIG. 4D) DIV7 neuronal cultures in a 384-well plate were treated with either vehicle or 10 mM KCl, and NLuc activities were measured 24 hours later. Each bar represents the NLuc activity in cells of each well. Error bars represent SEM.

FIGS. 5A-5D show HTS assay scale-up and assay reproducibility. LOPAC compounds were tested in 2 sets of 4 plates of cultured Bdnf^{NLuc/+} neurons. The activity of NLuc in each well is normalized to the average of NLuc activities in 1280 compound-containing wells. Red dash lines indicate 3× standard deviations (3×SDs) over the mean. Graphs show data from a single set of plates (FIGS. 5A and 5B) or the average of the two sets of plates (FIG. 5C). (FIG. 5D) The graph shows results from an independent screening of LOPAC compounds with 4 plates of cultured Bdnf^{NLuc/+} neurons. Red dash lines indicate 3×SDs over the mean. (FIG. 5E) Bay K8644 (10 μM) or vehicle (DMSO) was added to Bdnf^{NLuc/+} neuronal cultures at DIV7 for 24 hours. Unpaired t test, ****p<0.0001, n=12 wells/condition. Identified compounds (numbered points above dotted line in FIGS. 5A-5D): 1, brefeldin A; 2, Bay K8644; 3, 1,3,5-Tris(4-hydroxyphenyl)-4-propyl-1H-pyrazole; 4, nitrendipine; 5, nimodipine; 6, N-propargyl nitrendipine; 7, GR127935-HCl; 8, gabazine; 9, phorbol-12-myristate 13-acetate; 10, phenylbutazone; 11, PK11195; 12, felodipine.

FIG. 6 shows the chemical structure of GR127935-HCl (identified in the HTS assay above) and expression of Bdnf mRNA and four major Bdnf mRNA variants in DIV7 Bdnf^{NLuc/+} neurons following 24 hours of treatment with 5 μM GR127935-HCl, as assessed by quantitative RT-PCR (qRT-PCR). Two-tailed unpaired t test: *p<0.05 and **p<0.01. n=5.

### DEFINITIONS

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

An “open reading frame” or “ORF” is a portion of a DNA which contains a sequence of bases that could potentially encode a protein. As an example, an ORF can be located between the start-code sequence (initiation codon) and the stop-codon sequence (termination codon) of a gene. The term “in frame” refers to coding sequences that are part of the same ORF and could be translated continuously, e.g., as a fusion peptide, propeptide, prepropeptide, etc.

The term “genomically integrated” refers to a nucleic acid that has been introduced into a cell such that the nucleotide sequence is integrated into the genome of the cell and is capable of being inherited by progeny thereof. Any method suitable for stable integration of a nucleic acid into the genome of a cell may be used to form a genomically integrated nucleic acid.

The term “plasmid” or “vector” includes any known vector including a bacterial vector, a viral vector, an episomal plasmid, an integrative plasmid, or a phage vector. The term “vector” refers to a construct which is capable of delivering, and, optionally, expressing, one or more expressible sequences (e.g., protein coding sequence) in a host cell.

The term “targeting vector” refers to a vector that can be introduced by homologous recombination, non-homologous-end-joining-mediated ligation, or any other means of recombination to a target position in the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous” refers to a nucleic acid sequence that occurs naturally within a cell or rodent (e.g., a mouse). For example, an endogenous Bdnf sequence of a rodent refers to a native Bdnf sequence that naturally occurs at the Bdnf locus in the rodent.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two portions that do not naturally occur together in the same molecule. A heterologous sequence can be a sequence which is present in a cell, genome, or gene in the genetic context other than where it naturally occurs. For example, the term “heterologous,” when used with reference to portions of a nucleic acid or portions of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. A heterologous sequence can be a sequence derived from the same gene and/or cell type, but introduced into the cell or a similar cell in a different context, such as on an expression vector or in a different chromosomal location or with a different promoter. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas9 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, a “Bdnf locus” may refer to the specific location of a Bdnf gene, Bdnf DNA sequence, BDNF-encoding sequence, or Bdnf position on a chromosome of the genome of an organism that has been identified as to where such a sequence resides. A “Bdnf locus” may comprise a regulatory element of a Bdnf gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). The term “gene” also includes other non-coding sequences including regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “variant” refers to a nucleotide sequence differing from the sequence most prevalent in a population (e.g., by at least one nucleotide) or a protein sequence different from the sequence most prevalent in a population (e.g., by at least one amino acid).

The term “fragment” when referring to a protein means a protein that is shorter or has fewer amino acids than the full-length protein. The term “fragment” when referring to a nucleic acid means a nucleic acid that is shorter or has fewer nucleotides than the full-length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment.

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “isolated” with respect to proteins and nucleic acid refers to proteins and nucleic acids that are relatively purified with respect to other bacterial, viral, or cellular components that may normally be present in situ, up to and including a substantially pure preparation of the protein and the polynucleotide. The term “isolated” also includes proteins and nucleic acids that have no naturally occurring counterpart, have been chemically synthesized and are thus substantially uncontaminated by other proteins or nucleic acids, or has been separated or purified from most other cellular components with which they are naturally accompanied (e.g., other cellular proteins, polynucleotides, or cellular components).

The term “in vitro” includes artificial environments and to processes or reactions that occur within an artificial environment (e.g., a test tube). The term “in vivo” includes natural environments (e.g., organism or body) and to processes or reactions that occur within a natural environment. The term “ex vivo” includes cells that have been removed from the body of an individual and to processes or reactions that occur within such cells.

The term “reporter gene” refers to a nucleic acid having a sequence encoding a gene product (e.g., an enzyme or a detectable protein; e.g., a fluorescent protein) that is easily and quantifiably assayed when a construct comprising the reporter gene sequence operably linked to a heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, luciferase genes (e.g., nano luciferase), genes encoding beta-glucuronidase (GUS), and genes encoding fluorescent proteins. A “reporter protein” refers to a protein encoded by a reporter gene.

An “RNA-guided DNA endonuclease” is an enzyme (endonuclease) that uses RNA-DNA complementarity to identify target sites for sequence-specific double-stranded DNA (dsDNA) cleavage. An RNA-guided DNA endonuclease may be, but is not limited to, a zCas9 nuclease, a Cas9 nuclease, type II Cas nuclease, an nCas9 nuclease, a type V Cas nuclease, a Cas12a nuclease, a Cas12b nuclease, a Cas12c nuclease, a CasY nuclease, a CasX nuclease, a Cas12i nuclease, or an engineered RNA-guided DNA endonuclease.

A “guide RNA” (gRNA) comprises an RNA sequence (tracrRNA) bound by Cas and a spacer sequence (crRNA) that hybridizes to a target sequence and defines the genomic target to be modified. The tracrRNA and crRNA may be linked to form a “single chimeric guide RNA” (sgRNA).

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339:823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN [A/C/T]RRT, NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

The term “recombination” includes any process of exchange of genetic information between two polynucleotides and can occur by any mechanism. Recombination in response to double-strand breaks (DSBs) occurs principally through two conserved DNA repair pathways: non-homologous end joining (NHEJ) and homologous recombination (HR). See Kasparek & Humphrey (2011) Seminars in Cell & Dev. Biol. 22:886-897, herein incorporated by reference in its entirety for all purposes. Likewise, repair of a target nucleic acid mediated by an exogenous donor nucleic acid can include any process of exchange of genetic information between the two polynucleotides.

Recombination can occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLOS ONE 7: e45768: 1-9; and Wang et al. (2013) Nat Biotechnol. 31:530-532, each of which is herein incorporated by reference in its entirety for all purposes.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

Designation of a range of values includes all integers within or defining the range, and all subranges defined by integers within the range.

In general, the term “about” indicates variation in a quantity of a component of a composition not having a significant effect on the activity or stability of the composition. For example, “about” can mean within 1 standard deviation. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed. When the specification discloses a specific value for a parameter, the specification should be understood as alternatively disclosing the parameter at “about” that value. All ranges are to be interpreted as encompassing the endpoints in the absence of express exclusions, such as “not including the endpoints”; thus, for example, “within 10-15” or “from 10 to 15” includes the values 10 and 15.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

### DETAILED DESCRIPTION

### I. Overview

Disclosed herein are rodents and populations of rodent cells comprising a genetically modified Bdnf locus and methods of generating and using such rodents and rodent cells. Rodents and rodent cells comprising the genetically modified Bdnf locus express a BDNF-reporter (e.g., nano luciferase (NLuc)) fusion protein with expression and function that approximates wild type BDNF.

Current methods of determining BDNF levels in tissues or cultured neurons, including Western blot and ELISA, are too slow and expensive for high throughput screening (HTS). In contrast, the described genetically modified rodents and rodent cells express a BDNF-NLuc fusion that provides for sensitive and quantitative assays for BDNF detection that are amenable to HTS and are more cost and time effective. In addition, the described rodent and rodent cells, which produce a biologically active BDNF can report changes beyond just activation of transcription, including translation, stability, and/or secretion.

### II. Brain-Derived Neurotrophic Factor

Brain-derived neurotrophic factor (BDNF), a 28-kD dimeric secreted growth factor, binds to TrkB receptor tyrosine kinase, inducing its dimerization and activation. This activates many signaling cascades that cooperatively promote neuronal survival, regulate the development of neural circuits, stimulate synapse formation, enhance synaptic transmission, and facilitate synaptic plasticity in many brain regions. These crucial physiological functions implicate BDNF deficiency as a potential cause for several neuropsychiatric and neurodegenerative disorders, including Alzheimer's disease (AD), Huntington's disease (HD), major depressive disorder (MDD), anxiety, eating disorders, and Rett syndrome, making restoring/elevating BDNF function an attractive therapeutic goal.

The Bdnf gene is transcribed from 9 distinct promoters, each of which drives transcription of a short 5′ exon alternatively spliced onto a common 3′ exon (Exon 10) encoding the full length preproBDNF protein. Furthermore, Bdnf mRNAs transcribed from each promoter are polyadenylated at two alternative sites, leading to two distinct populations of mRNAs: those with a short 3′ untranslated region (UTR) and those with a long 3′ UTR. Thus, BDNF synthesis is regulated at levels of transcription and translation. Due to multiple promoters and alternative polyadenylation, the Bdnf gene produces 18 mRNA variants encoding the same BDNF protein, and distinct combinations of Bdnf mRNA variants are expressed in different tissues or brain regions. The usage of various BDNF promoters in humans differs in brain versus peripheral tissues. Transcripts from promoters 1, 2, 4 and 5 are abundant in the brain, but are low or absent in peripheral tissues.

Mouse Bdnf maps to 2 E3; 2 56.63 cM on chromosome 2 (NCBI RefSeq Gene ID 12064; assembly GRCm39 (GCF_000001635.27); location NC_000068.8 (109505045 . . . 109557388). The wild type mouse BDNF protein has been assigned UniProt accession number P21237. At least two isoforms are known (P21237-1 and P21237-2). The sequence for the canonical isoform, P21237-1 (identical to NCBI Accession Nos. NP_001041604.1, NP_001041606.1, NP_001041607.1, NP_001272345.1, NP_001272346.1, NP_001272347.1, NP_001272348.1, NP_001272349.1, NP_001272350.1, NP_001272351.1, and NP_001303239.1), is set forth in SEQ ID NO: 3. An exemplary mRNA (DNA) isoform encoding the canonical isoform is assigned NCBI Accession No. NM_001048139.1 and is set forth in SEQ ID NO: 1. An exemplary coding sequence (CDS) (CCDS ID CCDS38194.1) is set forth in SEQ ID NO: 2. The canonical full-length mouse preproBDNF protein set forth in SEQ ID NO: 3 has 249 amino acids, including a signal peptide (amino acids 1-18), a propeptide (amino acids 19-130), and a mature peptide (amino acids 131-249). Delineations between these domains are as designated in UniProt. Reference to mouse BDNF includes the canonical (wild type) forms as well as all allelic forms and isoforms. Any other forms of mouse BDNF have amino acids numbered for maximal alignment with the wild type form, aligned amino acids being designated the same number.

Rat Bdnf maps to 3q34 on chromosome 3 (NCBI RefSeq Gene ID 24225; assembly mRatBN7.2 (GCF_015227675.2); location NC_051338.1 (96165042 . . . 96215621). The wild type rat BDNF protein has been assigned UniProt accession number P23363. The sequence for P23363 (identical to NCBI Accession Nos. NP_001257560.1, NP_001257561.1, NP_001257562.1, NP_001257563.1, NP_001257564.1, NP_001257565.1, NP_001257566.1, and NP_001257567.1), is set forth in SEQ ID NO: 6. An exemplary mRNA (DNA) isoform is assigned NCBI Accession No. NM_001270631.1 and is set forth in SEQ ID NO: 4. An exemplary CDS is set forth in SEQ ID NO: 5. The canonical full-length rat preproBDNF protein set forth in SEQ ID NO: 6 has 249 amino acids, including a signal peptide (amino acids 1-18), a propeptide (amino acids 19-130), and a mature peptide (amino acids 131-249). Delineations between these domains are as designated in UniProt. Reference to rat BDNF includes the canonical (wild type) forms as well as all allelic forms and isoforms. Any other forms of rat BDNF have amino acids numbered for maximal alignment with the wild type form, aligned amino acids being designated the same number.

### III. Rodents Comprising a BDNF-NLuc Fusion Protein and Cells Derived Thereof

Rodents (e.g., mice, rats, hamsters, and guinea pigs) comprising the described endogenous Bdnf locus genetically modified to express a BDNF-NLuc fusion protein described above are provided. In certain aspects, the NLuc coding sequence is inserted in frame before the Bdnf stop codon (e.g., within about 10 codons). In further aspects, the NLuc coding sequence is flanked by a 5′ homology arm that contains the 850-bp sequence upstream of the stop codon, and a 3′ homology arm that contains the 803-bp sequence downstream of the stop codon. In still further aspects, silent mutations are introduced into the PAM motifs within the 5′ homology arm in the donor to block further Cas9 targeting and recutting. Other reporter proteins are known in the art and can readily substituted for the Nluc in the BDNF-NLuc fusion.

The rodents and cells derived from those rodents can be male or female. The rodents and cells derived from those rodents can be heterozygous or homozygous for the genetically modified Bdnf locus. A diploid organism has two alleles at each genetic locus. Each pair of alleles represents the genotype of a specific genetic locus. Genotypes are described as homozygous if there are two identical alleles at a particular locus and as heterozygous if the two alleles differ.

The rodents can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 129S5, 129S9/SvEvH, 129S6 (129/SvEvTac), 129S7, 129S8, 129T1, and 129T2. See, e.g., Festing et al. (1999) Mammalian Genome 10:836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RTI^{av1} haplotype. Such strains are available from a variety of sources including Harlan Laboratories. The Dark Agouti (DA) rat strain is characterized as having an agouti coat and an RTI^{av1} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

Various methods can be used to generate the targeted genetic modification in the polynucleotide or rodent genome of interest. Methods of obtaining the targeted genetic modifications as described in the instant application can comprise unaided homologous recombination, recombinase-based insertion, and DNA repair-based insertion and are known in the art (Dong et al., 2021, PNAS. 118 (22) e2004834117). Recombinase-based insertion can comprise systems and constructs involving site-specific recombinases, including but not limited to, Cre:loxP systems, Flp:FRT systems, Dre:rox systems, VCre:loxV systems, Gin:gix systems, Bxb1:attP/attB systems, phiC31:attP/attB systems. DNA repair-based insertion methods rely upon the activity of a nuclease agent, including but not limited to a Transcription Activator-Like Effector Nuclease (TALEN; see, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi: 10.1093/nar/gkq704; and Miller et al. (2011) Nature Biotechnology 29:143-148; all of which are herein incorporated by reference), a zinc-finger nuclease (ZFN; see, US20060246567; US20080182332; US20020081614; US20030021776; WO/2002/057308A2; US20130123484; US20100291048; WO/2011/017293A2; and Gaj et al. (2013) Trends in Biotechnology, 31 (7): 397-405 each of which is herein incorporated by reference), a meganuclease (see, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764), or a CRISPR/Cas system described elsewhere herein.

Any nuclease agent that induces a nick or double-strand break into a desired recognition site can be used in the methods and compositions disclosed herein. A naturally occurring or native nuclease agent can be employed so long as the nuclease agent induces a nick or double-strand break in a desired recognition site. In some aspects, the desired recognition site is near the stop codon of the targeted gene. Alternatively, a modified or engineered nuclease agent can be employed. An “engineered nuclease agent” includes a nuclease that is engineered (modified or derived) from its native form to specifically recognize and induce a nick or double-strand break in the desired recognition site. Thus, an engineered nuclease agent can be derived from a native, naturally occurring nuclease agent or it can be artificially created or synthesized. In some aspects, the engineered nuclease induces a nick or double-strand break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA. Assays to measure the double-strand break of a recognition site by a nuclease agent are known in the art (e.g., TaqMan® qPCR assay, Frendewey D. et al., Methods in Enzymology, 2010, 476:295-307, which is incorporated by reference herein in its entirety).

The length of the recognition site for insertion reporter into the Bdnf gene can vary, and includes, for example, recognition sites that are about 30-36 bp for a zinc finger nuclease (ZFN) pair (i.e., about 15-18 bp for each ZFN), about 36 bp for a Transcription Activator-Like Effector Nuclease (TALEN), or about 20 bp for a CRISPR/Cas9 guide RNA.

In some aspects, a BDNF-reporter (e.g., NLuc) fusion is created using a CIRSPR system. Described are nucleic acids for producing a genetically modified Bdnf locus using a CRISPR (e.g., CRISPR/Cas) system. The described nucleic acids can be used to target modification of the Bdnf locus and/or to insert or express one or more heterologous sequences (e.g., a reporter gene or fusion protein) in a rodent or rodent cell.

A CRISPR system comprises an RNA-guided DNA endonuclease enzyme and a CRISPR RNA. In some embodiments, a CRISPR RNA is part of a guide RNA. In certain aspects, the RNA-guided DNA endonuclease enzyme is a Cas9 protein. In some aspects, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the target gene genomic region. The Cas protein can be introduced into the rodent or rodent cell in the form of a protein or a nucleic acid (DNA or RNA) encoding the Cas protein (e.g., operably linked to a promoter expressible in the organism). The guide RNA can be introduced into the rodent or rodent cell in the form of RNA or a DNA encoding the guide RNA (e.g., operably linked to a promoter expressible in the organism). In some aspects, the CRISPR system further includes a DNA donor template.

Various methods and compositions are provided herein to allow for introduction of a CRISPR system targeting the Bdnf locus into a rodent or rodent cell. Methods for introducing nucleic acids into various cell types are known and include, for example, stable transfection methods, transient transfection methods, and virus-mediated methods.

Transfection protocols as well as protocols for introducing nucleic acid sequences into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. USA 74 (4): 1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, Sono-poration, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of a CRISPR system targeting the Bdnf locus into a rodent or rodent cell can also be mediated by electroporation, by intracytoplasmic injection, by viral infection, by adenovirus, by adeno-associated virus, by lentivirus, by retrovirus, by transfection, by lipid-mediated transfection, or by nucleofection. Introduction of a CRISPR system targeting the Bdnf locus into a rodent cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a protein or a polynucleotide encoding a protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a protein is injected into the cytoplasm and needs to be targeted to the nucleus, it can comprise a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. USA 107:15022-15026 and Meyer et al. (2012) Proc. Natl. Acad. Sci. USA 109:9354-9359.

The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

Guide sequences suitable for forming gRNAs or crRNAs for CRISPR system mediated genetic modification of a Bdnf locus are described. Suitable guide sequences include 17-23 nucleotide sequences along the Bdnf locus that are unique compared to the rest of the genome and immediately adjacent (5′) to a protospacer-adjacent motif (PAM) site. As an example, for the RNA-guided DNA endonuclease enzyme zCas9, a PAM site is NGG. Thus, any unique 17-23 nucleotide sequence immediately 5′ of a 5′-NGG-3′ in the Bdnf locus can be used in forming a gRNA. In some aspects, the guide sequence is 100% complementary to the target sequence. In other aspects, the guide sequence is at least 90% or at least 95% complementary to the target sequence. In further aspects, the guide sequence contains 0, 1, or 2 mismatches when hybridized to the target sequence. In still further aspects, a mismatch, if present, is located distal to the PAM, in the 5′ end of the guide sequence.

In certain aspects, the CRISPR system is designed to target the endogenous Bdnf locus. In further aspects, the CRISPR system is designed to insert one or more heterologous sequences (e.g., a donor template) into the Bdnf locus within 10, 9, 8, 7, 6, 5, 4, 3, 2, or 1 codon(s) of the stop codon of the Bdnf gene. In further aspects, the CRISPR system is designed to insert the one or more heterologous sequences within 1-5 codons, within 1-2 codons, or within 1 codon of the stop codon of the Bdnf gene. In further aspects, the CRISPR system is designed to insert the one or more heterologous sequences immediately before the stop codon of the Bdnf gene. In still further aspects, the CRISPR system is designed to insert a donor template that encodes nano luciferase (NLuc; CDS for NLuc provided in SEQ ID NO: 9) flanked by homology arms complementary to the endogenous Bdnf locus, such that NLuc is in frame with the Bdnf coding sequence and a fusion protein (BDNF-NLuc) is produced.

In some aspects, the 5′ homology arm contains a sequence upstream of the stop codon in exon 10 of the Bdnf gene, and the 3′ homology arm contains a sequence downstream of the stop codon in exon 10 of the Bdnf gene. The homology arms can be about 50 bp, about 100 bp, about 200 bp, about 300 bp, about 500 bp, about 1000 bp, about 1500 bp, or any length within this range. In some aspects, the homology arms are about 800 bp. In certain aspects, the 5′ homology arm contains an 850-bp sequence upstream of the stop codon, and the 3′ homology arm contains an 803-bp sequence downstream of the stop codon, as provided in SEQ ID NO: 7.

In some aspects, a spacer sequence is provided between the 5′ homology arm and the NLuc coding sequence. In further aspects, a spacer sequence is provided between the NLuc coding sequence and the 3′ homology arm. In still further aspects, the spacer sequences are about 3 bp, about 4 bp, about 5 bp, about 6 bp, about 7 bp, about 8 bp, about 9 bp, about 10 bp, about 15 bp, about 20 bp, about 25 bp, about 30 bp, about 40 bp, about 50 bp, or any length within this range. In certain aspects, the spacer between the 5′ homology arm and the NLuc coding sequence is CGGGATCCACCGGTCGCCACC (SEQ ID NO: 10) and the spacer between the NLuc coding sequence and the 3′ homology arm is AGATCT (SEQ ID NO: 11). In still further aspects, silent mutations are introduced into PAM motifs within the 5′ homology arm in the donor template to block further Cas9 targeting and recutting after homology-directed repair, as provided in SEQ ID NO: 7.

The DNA donor template can be provided a single strand DNA, double strand DNA, plasmid DNA, or viral vector DNA or RNA.

It is understood that RNA equivalents of any listed DNA sequences, substituting uracils (U) for thymines (T), may be used. An “RNA equivalent” is an RNA molecule having essentially the same complementary base pair hybridization properties as the listed DNA sequence.

CRISPR modification of the Bdnf locus is not limited to the CRISPR/zCas9 system. Other CRISPR systems using different nucleases and having different PAM sequence requirements are known in the art. PAM sequences vary by the species of RNA-guided DNA endonuclease. For example, Class 2 CRISPR-Cas type II endonuclease derived from S. pyogenes utilizes an NGG PAM sequence located on the immediate 3′ end of the guide sequence. Other PAM sequences include, but are not limited to, NNNNGATT (Neisseria meningitidis), NNAGAA (Streptococcus thermophilus), and NAAAAC (Treponema denticola). Guide sequences for CRISPR systems having nucleases with different PAM sequence requirements are identified as described above for zCas9, substituting the different PAM sequences.

Two or more guide RNAs can used with the same RNA-guided DNA endonuclease (e.g., Cas nuclease) or different RNA-guided DNA endonucleases.

Any of the above-described guide RNAs can be provided as an RNA or a DNA encoding the RNA.

In some aspects, a CRISPR system comprises one or more guide RNAs and a nucleic acid encoding an RNA-guided DNA endonuclease. In other aspects, a CRISPR system comprises one or more guide RNAs, a nucleic acid encoding an RNA-guided DNA endonuclease, and a DNA donor template.

In some aspects, a CRISPR system comprises a guide RNA and an RNA-guided DNA endonuclease in a complex. In other aspects, a CRISPR system comprises two or more guide RNAs, each in a complex with an RNA-guided DNA endonuclease. In still further aspects, a CRISPR system comprises a guide RNA and an RNA-guided DNA endonuclease in a complex and a DNA donor template.

The rodent cells derived from rodents comprising a genetically modified Bdnf locus can be any type of undifferentiated or differentiated state. For example, a cell can be a totipotent cell, a pluripotent cell (e.g., a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cells provided herein can also be germ cells (e.g., sperm or oocytes). The cells can be mitotically competent cells or mitotically inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be neurons, such as hippocampal neurons or cortical neurons.

Suitable cells provided herein also include primary cells. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture. Such cells can be isolated by conventional techniques and include, for example, hippocampal neurons or cortical neurons.

The cells provided herein also include one-cell stage embryos (i.e., fertilized oocytes or zygotes). Such one-cell stage embryos can be from any genetic background (e.g., BALB/c, C57BL/6, 129, or a combination thereof for mice), can be fresh or frozen, and can be derived from natural breeding or in vitro fertilization.

The cells provided herein can be normal, healthy cells, or can be diseased or mutant-bearing cells.

### IV. Methods of Screening BDNF Targeting Drugs In Vitro and In Vivo

The described rodents, or cells derived from the described rodents, can be used to identify molecules (possible Bdnf-modulating compounds) to target or alter the expression, activity, and/or stability of BDNF. The described rodents, or cells derived from the described rodents, can be used to analyze the effect of one or more molecules on BDNF expression, activity, and/or stability. The methods disclosed herein comprise introducing into a rodent comprising a genetically modified Bdnf locus encoding a BDNF-reporter fusion protein, or cells derived from these rodents, one or more molecules (e.g., possible BDNF-modulating compounds such as small molecules), including but not limited to nucleic acids, proteins, nucleic-acid-protein complexes, peptide mimetics, antigen-binding proteins, or small molecules, and observing or measuring the effect on expression and/or stability of the reporter. “Introducing” includes presenting to a rodent or a rodent cell the molecule (e.g., nucleic acid or protein or small molecule) in such a manner that it gains access to the interior of the cell or to the interior of cells within the rodent. The introducing can be accomplished by any means suitable for introducing a molecule into a cell. If multiple molecules are introduced, they can be introduced simultaneously or sequentially in any combination. In addition, two or more molecules can be introduced into a rodent or a rodent cell by the same delivery method or different delivery methods. Similarly, two or more molecules can be introduced into a rodent by the same route of administration or different routes of administration.

Described are methods of screening one or more molecules for modulation of Bdnf expression and/or stability comprising: delivering, in vitro, the one or more compounds to one or more cells derived from the described rodents comprising a genetically modified Bdnf locus encoding a BDNF-reporter fusion protein. The cells can be, but are not limited to, neuronal cells. In some aspects, neuronal cells are harvested from the brains of embryos of the described rodents. These embryos may be about embryonic day 15.5 (E15.5), about E16.5, about E17.5, about E18.5, about E19.5, about E20.5, or any age of embryonic development within this range. In certain aspects, cortices and hippocampi are harvested from E18.5 embryos comprising a genetically modified Bdnf locus encoding a BDNF-NLuc fusion protein to establish primary neuronal cell populations.

In some aspects, primary neuronal cell populations as described above are cultured for some time in vitro prior to the use of the neuronal cell populations in screening the one or more molecules. In some aspects, cells are plated into 96-well plates at about 2×10⁴ cells per well, about 3×10⁴ cells per well, about 4×10⁴ cells per well, about 5×10⁴ cells per well, about 6×10⁴ cells per well, or any number of cells per well within this range. In certain aspects, cells are plated in 96-well plates at about 4.5×10⁴ cells per well. In other aspects, cells are plated into 384-well plates at about 0.3×10⁴ cells per well, about 0.6×10⁴ cells per well, about 0.9×10⁴ cells per well, about 1.2×10⁴ cells per well, about 1.5×10⁴ cells per well, about 1.8×10⁴ cells per well, or any number of cells per well within this range. In certain aspects, cells are plated in 384-well plates at about 1×10⁴ cells per well.

In some aspects, the primary neuronal cell populations described above are plated in a defined plating media before being replaced with a defined feeding media. In some aspects, about 50%, about 60%, about 70%, about 80%, about 90%, about 100%, or any amount of plating media within this range is replaced with feeding media. In some aspects, plating media is replaced with feeding media about 12 hours, about 14 hours, about 16 hours, about 18 hours, about 20 hours, or any time within this range after plating. In certain aspects, 75% of plating media is replaced with feeding media 16 hours after plating.

Plating media may comprise Neurobasal (Gibco) media and any number of cell culture supplements, including, but not limited to penicillin-streptomycin (pen/strep, Gibco), fetal bovine serum (FBS, HyClone), B27 (Gibco), and/or GlutaMAX (Gibco). Plating media may comprise about 30 U/mL pen/strep, about 40 U/mL pen/strep, about 50 U/mL pen/strep, about 60 U/mL pen/strep, about 70 U/mL pen/strep, or any value within this range. Plating media may comprise about 1% FBS, about 2% FBS, about 3% FBS, about 4% FBS, about 5% FBS or any value within this range. Plating media may comprise about 0.5% B27, about 1% B27, about 1.5% B27, about 2% B27, about 2.5% B27, about 3% B27, or any value within this range. Plating media may comprise about 0.5 mM GLUTAMAX, about 1 mM GLUTAMAX, about 1.5 mM GLUTAMAX, about 2 mM GLUTAMAX, about 2.5 mM GLUTAMAX, about 3 mM GLUTAMAX, or any value within this range. In certain aspects, plating media comprises 2% FBS, 2% B27, 2 mM GlutaMAX, and 50 U/mL pen/strep.

Feeding media may comprise Neurobasal (Gibco) media and any number of cell culture supplements, including, but not limited to penicillin-streptomycin (pen/strep, Gibco), fetal bovine serum (FBS, HyClone), B27 (Gibco), GlutaMAX (Gibco) and/or 5-fluoro-2′-deoxyuridine (FUdR) to prevent overgrowth of non-neuronal cells. Feeding media may comprise about 30 U/mL pen/strep, about 40 U/mL pen/strep, about 50 U/mL pen/strep, about 60 U/mL pen/strep, about 70 U/mL pen/strep, or any value within this range. Feeding media may comprise about 1% FBS, about 2% FBS, about 3% FBS, about 4% FBS, about 5% FBS or any value within this range. Feeding media may comprise about 0.5% B27, about 1% B27, about 1.5% B27, about 2% B27, about 2.5% B27, about 3% B27, or any value within this range. Feeding media may comprise about 0.5 mM GLUTAMAX, about 1 mM GLUTAMAX, about 1.5 mM. GLUTAMAX, about 2 mM GLUTAMAX, about 2.5 mM GLUTAMAX, about 3 mM GLUTAMAX, or any value within this range. Feeding media may comprise about 10 μM FUdR, about 12 μM FUdR, about 14 μM FUdR, about 16 μM FUdR, about 18 μM FUdR, about 20 M FUdR, or any amount within this range. In certain aspects, feeding media comprises 2% FBS, 2% B27, 2 mM GlutaMAX, 50 U/mL pen/strep, and 16 μM FUdR.

Plating and media changes may be performed using a Versette liquid handler (Thermo Fisher Scientific), or the like, or an 8-channel pipette for the 384-well and 96-well plates, respectively.

In some aspects, cells are cultured at 37° C. and 5% CO₂ for about 3 days in vitro (DIV3), about DIV4, about DIV5, about DIV6, about DIV7, about DIV8, about DIV9, about DIV10, about DIV11, about DIV12, or any time within this range prior to performing a screening assay. In certain aspects, cells are cultured for DIV7 prior to performing a screening assay.

Introducing the BDNF-modulating molecule or possible BDNF-modulating molecule to the primary neuronal cell population as described above can be accomplished by any means suitable for introducing a molecule to a cell. In some aspects, the BDNF-modulating molecule (e.g., small molecule or protein) is delivered in a buffer (e.g., phosphate buffered saline; PBS) or solvent (e.g. dimethyl sulfoxide; DMSO) directly into the feeding media of the cell population. In other aspects, BDNF-modulating molecules (e.g., nucleic acids or proteins) are be introduced in a composition comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein. Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Introduction of BDNF-modulating molecules into a cell can also be mediated by electroporation, by intracytoplasmic injection, by viral infection, by adenovirus, by adeno-associated virus, by lentivirus, by retrovirus, by transfection, by lipid-mediated transfection, by nucleofection, or by hydrodynamic delivery (HDD).

Dosing or otherwise introducing BDNF-modulating molecules in vitro may be performed using Beckman Coulter's Biomek NXP automated liquid handler (Beckman), or the like, or an 8-channel pipette for the 384-well and 96-well plates, respectively.

Assessing the activity of BDNF-modulating molecules in the primary neuronal cell populations as described above can be accomplished by any standard molecular biology methodology to examine mRNA expression, protein expression, or enzymatic activity of the BDNF-Nluc fusion protein. In some aspects, luciferase activity is measured about 12 hours, about 18 hours, about 24 hours, about 30 hours, about 36 hours, or any time within this range after dosing with the BDNF-modulating molecule. In certain aspects, luciferase activity is measured 24 hours after dosing using the Nano-Glo Luciferase Assay System (Promega), or the like, and luminescence intensity iss measured using a microplate reader (FLUOstar Omega, BMG LABTECH), or the like, at 460 nm.

Described are methods by which BDNF-modulating compounds are screened in vivo in rodents comprising a genetically modified Bdnf locus encoding a BDNF-NLuc fusion protein, as described elsewhere herein. Administration (i.e., introduction of the BDNF-modulating molecule) in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. In a specific example, a BDNF-modulating molecule is administered via direct hippocampal injection, subcutaneous injection, or intravitreal injection.

Compositions comprising BDNF-modulating molecules can be formulated using one or more physiologically and pharmaceutically acceptable carriers, diluents, excipients, or auxiliaries. The formulation can depend on the route of administration chosen. The term “pharmaceutically acceptable” means that the carrier, diluent, excipient, or auxiliary is compatible with the other ingredients of the formulation and not substantially deleterious to the recipient thereof.

The frequency of administration and the number of dosages can depend on the half-life of the BDNF-modulating molecules and the route of administration, among other factors. The introduction of BDNF-modulating molecules into the rodent can be performed one time or multiple times over a period of time. For example, the introduction can be performed at least two times over a period of time, at least three times over a period of time, at least four times over a period of time, at least five times over a period of time, at least six times over a period of time, at least seven times over a period of time, at least eight times over a period of time, at least nine times over a period of time, at least ten times over a period of time, at least eleven times over a period of time, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time. Intervals may be regular or irregular based on response in the rodent, among other factors.

Rodents can be any relevant postnatal age in which assessing the activity of a BDNF-modulating molecule is desired.

In some aspects, assessing the activity of BDNF-modulating molecules in rodents comprising a genetically modified Bdnf locus encoding a BDNF-NLuc fusion protein is accomplished by in vivo bioluminescent imaging of the whole animal, or relevant region of interest in the animal (e.g., head or torso), following administration (e.g., intravenous or intraperitoneal injection) of a luciferase substrate. In some aspects, assessing the activity of BDNF-modulating molecules in rodents comprising a genetically modified Bdnf locus encoding a BDNF-NLuc fusion protein is accomplished in tissues isolated from these rodents or extracts of these isolated tissues. In some aspects, the activity of BDNF-modulating molecules is accomplished in tissues isolated from these rodents or extracts of these isolated tissues by any standard molecular biology methodology to examine mRNA expression, protein expression, or enzymatic activity of the BDNF-Nluc fusion protein. In a certain aspect, cortices and striata are isolated and homogenized using RIPA lysis buffer supplemented with protease inhibitors (cOmplete™ Mini, Sigma), and luciferase activity in protein extracts is detected using the Nano-Glo Luciferase Assay System (Promega), or the like, while luminescence intensity is measured using a microplate reader (FLUOstar Omega, BMG LABTECH), or the like, at 460 nm.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website, or the like are published at different times, the version most recently published at the effective filing date of the application is meant, unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases and one letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 1
Description of Sequences

| SEQ ID NO | Type | Description |
|---|---|---|
| 1 | DNA | Mouse Bdnf cDNA |
| 2 | DNA | Mouse Bdnf CDS |
| 3 | Protein | Mouse BDNF protein |
| 4 | DNA | Rat Bdnf cDNA |
| 5 | DNA | Rat Bdnf CDS |
| 6 | Protein | Rat BDNF protein |
| 7 | DNA | BDNF-NLuc donor template |
| 8 | Protein | BDNF-NLuc protein |
| 9 | DNA | NLuc CDS |
| 10 | DNA | 5′ homology arm spacer |
| 11 | DNA | 3′ homology arm spacer |
| 12 | DNA | Mouse Bdnf targeting sgRNA1 |
| 13 | DNA | Mouse Bdnf targeting sgRNA2 |
| 14 | DNA | PCR primer 1 |
| 15 | DNA | PCR primer 2 |
| 16 | DNA | PCR primer 3 |
| 17 | DNA | PCR primer 4 |
| 18 | DNA | PCR primer 5 |
| 19 | DNA | PCR primer 6 |
| 20 | DNA | PCR primer 7 |
| 21 | DNA | Bdnf coding forward primer |
| 22 | DNA | Bdnf coding reverse primer |
| 23 | DNA | Bdnf Exon I forward primer |
| 24 | DNA | Bdnf Exon I reverse primer |
| 25 | DNA | Bdnf Exon II forward primer |
| 26 | DNA | Bdnf Exon II, IV, and VI reverse primer |
| 27 | DNA | Bdnf Exon IV forward primer |
| 28 | DNA | Bdnf Exon VI forward primer |

### EXAMPLES

### Example 1. Generation of Bdnf^{NLuc} Knockin Mice

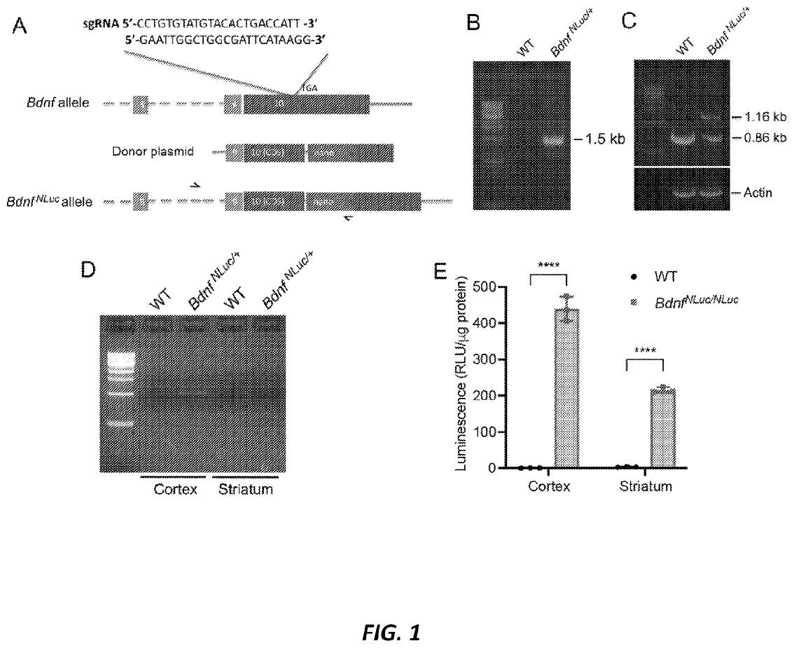

We employed CRISPR/Cas9 technology to generate a Bdnf^{NLuc} allele (FIG. 1A). We obtained 13 mice from injection of two sgRNAs and one donor plasmid. Using PCR, we identified 5 mice containing the expected NLuc insertion at the Bdnf locus (FIG. 1B). Of the 5 mice, two mice produced Bdnf^{NLuc/+} offspring after they were crossed to WT mice. As expected, Bdnf^{NLuc/+} mice produced both Bdnf mRNA and Bdnf-NLuc mRNA (FIG. 1C). Also, Bdnf-NLuc mRNA was detected in the cortex of Bdnf^{NLuc/+} mice but not in the WT cortex or in the Bdnf^{NLuc/+} striatum where the Bdnf gene is not expressed (Altar et al., 1997) (FIG. 1D). These RT-PCR results indicate that the Bdnf Luc allele is expressed and regulated in the same way as a normal Bdnf allele. We detected NLuc activity in cortical lysates of Bdnf^{NLuc/-} mice using Promega Nano-Glo Luciferase Assay System (FIG. 1E). We also detected NLuc in the striatal lysates of Bdnf^{NLuc/+} mice (FIG. 1E), indicating that BDNF-NLuc is transported to the striatum from BDNF-expressing afferent neurons as does BDNF.

Bdnf^{NLuc/NLuc} mice grew normally, did not show aggression like Bdnf/heterozygous mice (Lyons et al., 1999), became heavier than WT mice at 11 weeks of age (32.1±1.9 g vs. 27.0±0.7 g for male mice; p=0.033), and were fertile. These phenotypes indicate that the BDNF-NLuc fusion protein functions nearly as efficiently as BDNF in the nervous system.

### Example 2. Culturing Bdnf^{NLuc+} Neurons in 384-Well Microplates

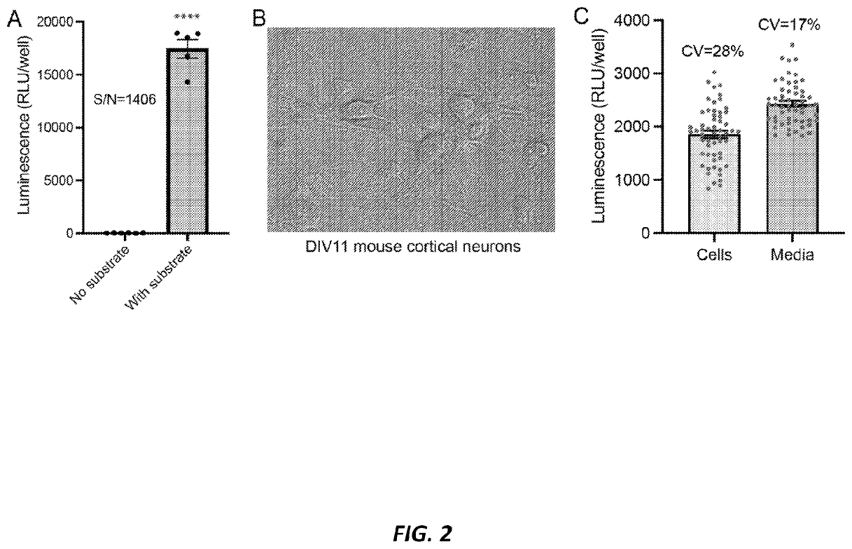

The first step toward high-throughput screening (HTS) assay development is to establish neuronal cultures in a miniaturized format. We could establish cultures of neurons isolated from cortices and hippocampi of newborn Bdnf^{NLuc/+} pups in both 96- and 384-well plates (FIGS. 2A and 2B). We selected the cortex and hippocampus as the source of primary neurons for their relevance to many CNS disorders and their richness in BDNF-expressing neurons. We could obtain approximately 1×10⁷ cells from the cortex and hippocampus of one newborn pup, which is sufficient to seed a 96-well plate (~50,000 cells/well) or a 384-well plate (~20,000 cells/well). The assay for NLuc activity in cultured Bdnf^{NLuc+} neurons was highly specific, producing a signal-to-noise ratio (S/N) of 1406 because neurons did not emit any luminescence in the absence of the NLuc substrate furimazine (FIG. 2A). The assay was also highly sensitive, as Bdnf^{NLuc/+} neurons cultured in 384-well plates still produced robust luminescence (FIG. 2C). As BDNF is a secreted protein, BDNF-NLuc was detected in both cells and media (FIG. 2C).

### Example 3. Optimization of Bdnf^{NLuc/+} Neuronal Assay for HTS

We next optimized the assay through variation of culture conditions. We have established a procedure resulting in consistent assay reliability and reproducibility. The procedure includes culturing Bdnf^{NLuc/+} neurons isolated from cortices and hippocampi of embryos (from WT×Bdnf^{NLuc/NLuc} crosses) at embryonic day 18.5, using a liquid handling device to plate cells and handle other solutions in 384-well plates, plating 10,000 cells/well, plating cells with a Neurobasal medium containing 2% fetal bovine serum and 2% B27, then replacing 75% of the plating medium with a Neurobasal medium containing 2% B27 a day later, and adding the thymidylate synthase inhibitor FUdR to media to kill rapidly dividing cells.

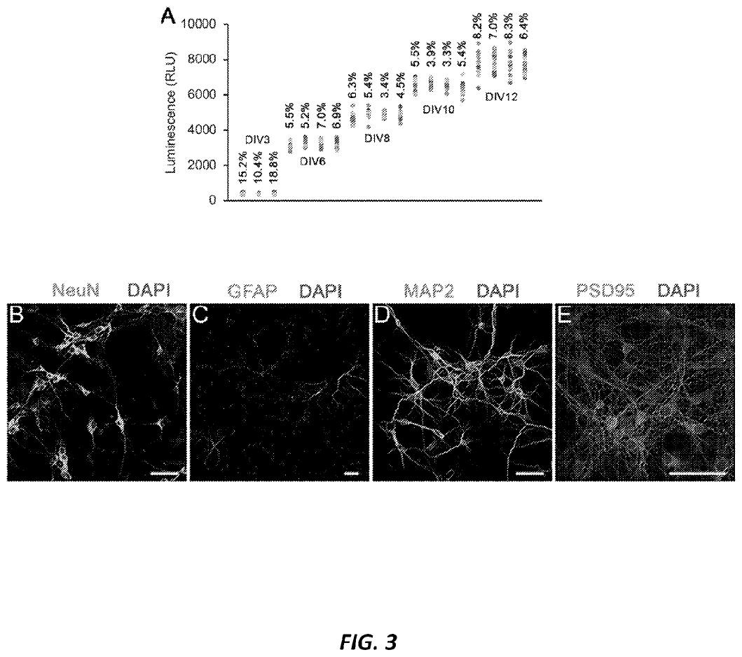

Using this new procedure, we could consistently establish healthy cultures of Bdnf^{NLuc/+} neurons. To determine an age when the Bdnf^{NLuc/+} neuronal culture is most suitable for compound screening, we measured the NLuc activity in media from cultures on DIV3, DIV6, DIV8, DIV10, and DIV12 (FIG. 3A). Well-to-well variation in NLuc activity in cultures on DIV6-DIV10 was small with a CV of ~5%. Furthermore, cells in Bdnf^{NLuc/+} cultures on DIV7 were mostly neurons (FIG. 3B) with long dendrites (FIG. 3D) many synapses (FIG. 3E), and only a small number of them were astrocytes (FIG. 3C). These results indicate that we can treat Bdnf^{NLuc/+} cultures on DIV7 to determine if a compound has potential to alter BDNF expression in neurons.

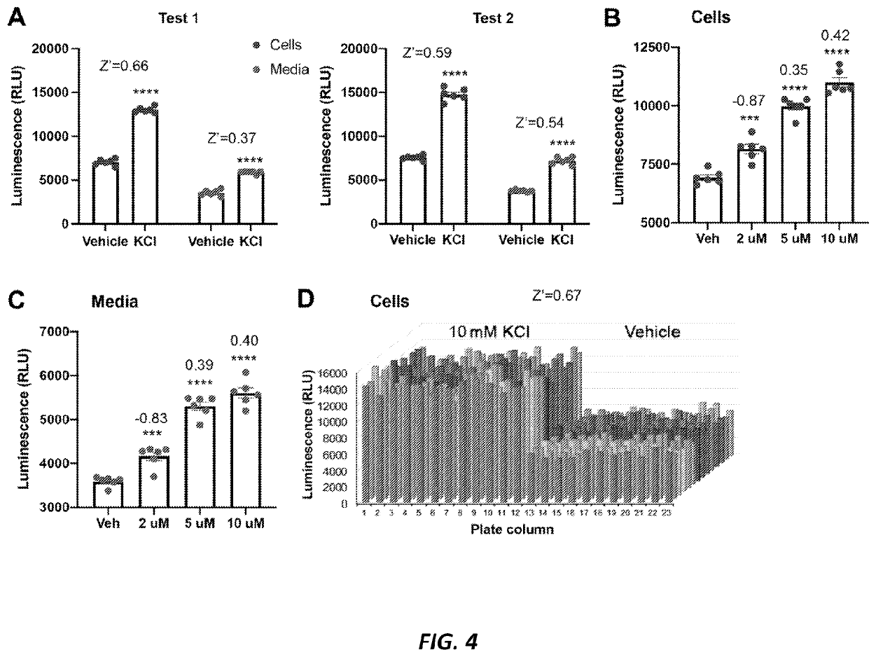

We tested our assay for suitability for HTS by using two positive controls. Several chemicals have been reported to stimulate Bdnf gene expression, including KCl (Ghosh et al., 1994; Tao et al., 2002) and insecticide deltamethrin (DM) (Takasaki et al., 2013). Membrane depolarization induced by incubation with KCl at 50 mM for 90 min increases Bdnf gene expression in cultured neurons for a short duration (Ghosh et al., 1994; Tao et al., 2002). We found that KCl at a final concentration of 10 mM elevated levels of BDNF in both cell bodies and media in our neuronal cultures over 24 hours without detectable cytotoxicity (FIG. 4A). DM also stimulated BDNF production in our neuronal cultures in a dose-dependent manner (FIGS. 4B and 4C). The assay produces a good Z′ for both 10 mM KCl and 10 μM DM (FIGS. 4A-4C) (Zhang et al., 1999). To measure NLuc activities in cells, we can simply invert a plate to dump culture medium and then add the Promega Nano-Glo mix, whereas we must transfer a certain amount of culture medium to a new plate and then add the Promega Nano-Glo mix to measure NLuc activities in media.

To determine assay reproducibility throughout the 384-well plate, we treated half of a plate of neuronal cultures with vehicle and the other half with 10 mM KCl for 24 hours, and the assay produced a Z′ of 0.67 (FIG. 4D). The Z′ for DM is consistently lower than that of KCl (FIG. 4), likely because DM is a less potent inducer of BDNF expression. Our NLuc assay in cells for 10 mM KCl has a reproducible Z′>0.50 (FIG. 4), a measure considered suitable for HTS (Zhang et al., 1999).

### Example 4. Scalability and Repeatability of the Bdnf^{NLuc/+} Phenotypic Assay

We tested the scalability of the Bdnf^{NLuc/+} phenotypic assay by screening the 1280-compound Library of Pharmacologically Active Compounds (LOPAC). We dissected out cortices and hippocampi of 8 Bdnf^{NLuc/+} embryos at E18.5 from 2 timed pregnant mice, prepared a suspension of dissociated neurons (10,000 cells/80 μl), transferred 40 ml of the suspension into each of eight 50-ml Falcon centrifuge tubes, used the liquid handling device to plate cells from each aliquot of 40-ml cell suspension onto a 384-well plate (80 μl/well), and cultured neurons.

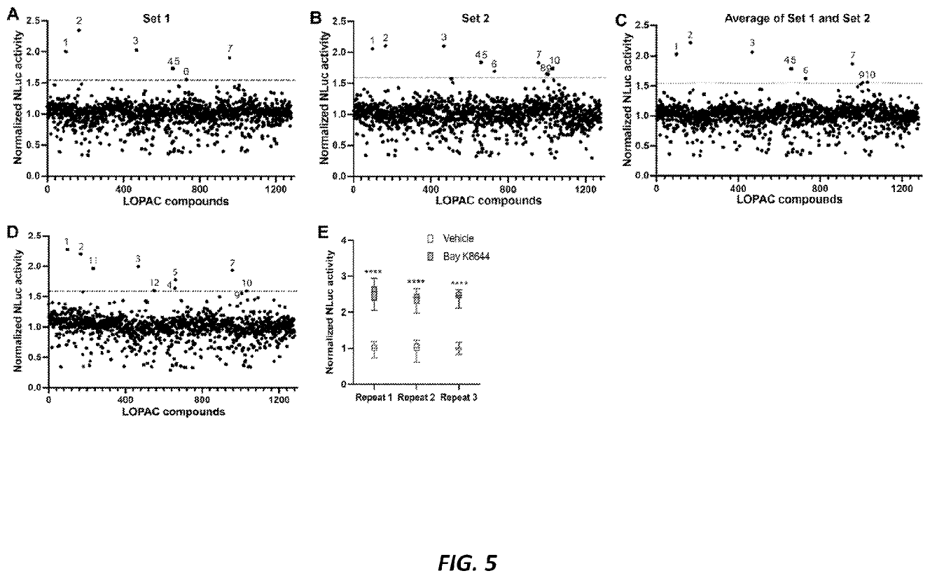

Using 2 sets of 4 plates each of DIV7 neuronal cultures, we removed 20 μl of medium from each well and then added each LOPAC compound in 20 μl of fresh medium to a well to reach a final concentration of 10 μM. LOPAC compounds (320) were added to wells in the middle 20 columns of each plate, while the first 2 and last 2 columns were used for vehicle controls and positive controls. After 24 hours of incubation, we inverted plates to dump culture medium and then measured NLuc activity in neurons. The NLuc activity in each well is normalized to the average of NLuc activities in 1280 compound-containing wells. The intra-assay CV between 2 sets of plates was 5.9% and the 8 plates had an average Z′ of 0.61±0.04 for positive control Bay K8644, indicating excellent plate-to-plate assay repeatability. Compounds that increased the NLuc activity to more than 3× standard deviations (3×SDs) over the mean were considered a hit, and in this case, there are 7 hits in the first set of plates (FIG. 5A) and 10 hits in the second set of plates (FIG. 5B), 7 of which are shared. When the average of duplicate assays for each compound is plotted, we identify the same hits from the second set of plates, with the exception of hit 8 (FIG. 5C). To test the repeatability of the assay in different batches of cultured neurons, we repeated the screening with 4 different plates of cultured neurons. The repeat screening rediscovered almost all hits that were found in the original screening (hits 1, 2, 3, 4, 5, 7, and 10) except hits 6 and 9 (FIG. 5D; hit 9 is just under the threshold). To further test the reproducibility of the assay, we examined Bay K8644 in 3 batches of cultured neurons prepared on different weeks and found that Bay K8644 increased BDNF levels to a similar extent in the 3 trials (FIG. 5E). These results demonstrate high scalability and repeatability of the assay.

The hits identified in the above screening include brefeldin A, L-type voltage-gated calcium channel (L-VGCC) agonist (Bay K8644), L-VGCC blockers (nitrendipine, nimodipine, N-propargyl nitrendipine, & felodipine), estrogen receptor alpha agonist [(1,3,5-Tris(4-hydroxyphenyl)-4-propyl-1H-pyrazole] (PPT), 5-HT1B and 5-HT1D antagonist (GR127935 hydrochloride hydrate), protein kinase C activator (phorbol-12-myristate 13-acetate), and cyclooxygenase inhibitor (phenylbutazone). In further support of the validity of the HTS assay, several of these hits are supported by previous reports in the literature. Brefeldin A was shown to inhibit protein transport from the ER to the Golgi complex (Helms and Rothman, 1992), so that it can increase intracellular BDNF levels by inhibiting BDNF secretion. KCl stimulates Bdnf gene expression by activating Ca²⁺ influx through L-VGCC (Ghosh et al., 1994), and thereby Bay K8644 stimulates Bdnf gene expression in neurons by opening L-VGCC (Zafra et al., 1992). Phorbol myristate acetate was reported to enhance the effect of Bay K8644 on Bdnf gene expression (Zafra et al., 1992).

### Example 5. Validation of Novel HTS-Identified Hit on Bdnf mRNA Expression in Neurons

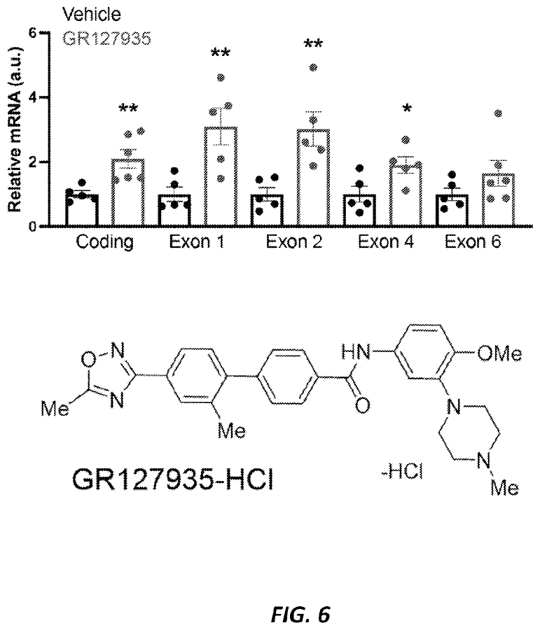

GR127935-HCl (FIG. 6) is a potent and selective 5-HT1B/1D receptor antagonist that has not previously been reported to stimulate Bdnf gene expression. We treated DIV7 Bdnf^{NLuc/+} neurons in a 24-well plate with 5 μM GR127935-HCl for 24 hours, isolated total RNA from cultured cells in each well, and ran quantitative RT-PCR (qRT-PCR) to determine levels of total Bdnf mRNA (coding), as well as four major Bdnf mRNA variants (exon 1, exon 2, exon 4, and exon 6). We found that GR127935-HCl elevated Bdnf mRNA levels by stimulating transcription from exon 1, exon 2, and exon 4 promoters (FIG. 6).

### Materials and Methods

Animals. Wild type C57BL/6J mice were purchased from the Jackson Laboratory. All mice were given free access to food and water and housed in a 12 h light/12 h dark cycle. All procedures described here were approved by the Institutional Animal Care and Use Committee at UF Scripps Biomedical Research and were in compliance with the National Institutes of Health guide for the care and use of laboratory animals.

Generation of Bdnf^{Nluc} allele. We generated a Bdnf′^{Nluc} mouse allele by inserting the NLuc-coding sequence immediately before the stop codon at the Bdnf locus using CRISPR/Cas9 techniques. sgRNAs were designed using the CRISPR tool (http://crispr.mit.edu) to minimize potential off-target effects. Two sgRNAs were selected (sgRNA1: 5′-CCTGTGTATGTACACTGACCATT-3′ [SEQ ID NO: 12] and sgRNA2: 5′-GAATTGGCTGGCGATTCATAAGG-3′ [SEQ ID NO: 13]) for integration of the NLuc sequence. The DNA donor plasmid contains two homology arms flanking the NLuc sequence, which were cloned into BamHI digested pBluescript II KS (−) vector using the Gibson assembly method (New England Biolabs, E5510). The 5′ homology arm contains the 850-bp sequence upstream of the stop codon, whereas the 3′ homology arm contains the 803-bp sequence downstream of the stop codon. To block further Cas9 targeting and recutting after undergoing homology-direct repair, we introduced silent mutations into the PAM motifs within the 5′ homology arm in the donor plasmid by using Q5 Site-Directed Mutagenesis kit (New England Biolabs, E0054). The donor plasmid (template) was confirmed with DNA sequencing and is represented by SEQ ID NO: 7. Microinjection of mixture of sgRNAs, donor DNA, and Cas9 protein was performed by the Genomic Modification Facility at Scripps Research. Zygotes were cultured to the blastocyst stage in vitro. Genomic DNA from blastocysts was extracted and a PCR screen was performed to select blastocysts with successful homologous recombination. Positive blastocysts were transferred into the oviducts of pseudo-pregnant females to produce the founder mice.

Primary cortical/hippocampal cell culture and maintenance. Brains of E18.5 Bdnf^{NLuc/+} embryos were collected and placed in ice-cold Ca²⁺ and Mg²⁺ free Hanks' balanced salt solution (HBSS) supplemented with Penicillin-Streptomycin (Pen/Strep, 50 U/mL, Gibco), 25 mM D-glucose, 1 mM pyruvate, and 20 mM HEPES. Cortices and hippocampi were isolated and digested with papain (~12 U/mL, Worthington) at 37° C. for 20 min. Brain tissues were washed with media containing Neurobasal (Gibco), 4% fetal bovine serum (FBS, HyClone), and Pen/Strep. The tissue was gently triturated and allowed to settle, and the supernatant was filtered through a 40 μm cell strainer. Cells were centrifuged at 420×g for 4 min and resuspended in plating media [Neurobasal, 2% FBS, 2% B27 (Gibco), 2 mM GlutaMAX (Gibco), and Pen/Strep]. 1.0×10⁴ or 4.5×10⁴ cells were plated onto white Greiner μClear 384-well plate (80 μl/well) or PDL-coated 96-well plate (200 μl/well), respectively. Sixteen hours after plating, 75% of the plating media was replaced by feeding media [Neurobasal, 2% B27, 2 mM GlutaMAX, and Pen/Strep] supplemented with 16 μM 5-fluoro-2′-deoxyuridine (FUdR) to prevent overgrowth of non-neuronal cells. Plating and media changes were performed using a Versette liquid handler (Thermo Fisher Scientific) or an 8-channel pipette for the 384-well and 96-well plates, respectively. Cells were maintained at 37° C. and 5% CO₂ incubator.

Compound treatment and LOPAC library screening. Compound treatment or library screening in primary cultures of Bdnf^{NLuc/+} mouse neurons on day 7 in vitro (DIV7) was performed by replacing one-quarter of the culture medium with equal volume of compound-containing medium to yield the desired final concentration for each compound. For the library of pharmacologically active compounds (LOPAC, Millipore), compounds in the original source plates were first dosed using a pintool into 384-well plates containing feeding medium to create a secondary source plate with compounds at 40 μM using the Beckman Coulter's Biomek NXP automated liquid handler (Beckman). 1.6% DMSO and 40 μM (+)-Bay K8644 (Sigma) were added to the first two and the last two columns of the secondary source plate as vehicle and positive control, respectively. Compounds in the secondary source plates were then added to culture neurons in the 384-well plates using a Versette liquid handler. Neurons were treated with compounds for 24 hours in a humidified incubator set to 37° C. and 5% CO₂. Luciferase activity was detected the next day using Nano-Glo Luciferase Assay System (Promega) according to the manufacturer's instruction. Luminescence intensity was measured using a microplate reader (FLUOstar Omega, BMG LABTECH) at 460 nm.

qRT-PCR validation of HTS hit. DIV7 Bdnf^{NLuc/+} neurons were treated in a 24-well plate with 5 μM GR127935-HCl for 24 hours, after which total RNA was isolated from cultured cells in each well and used to perform quantitative RT-PCR (qRT-PCR) to determine the levels of total Bdnf mRNA (coding), as well as four major Bdnf mRNA variants (exon 1, exon 2, exon 4, and exon 6). Levels of Bdnf mRNA variants were quantified using qRT-PCR with 18S rRNA as the internal control. Primer sequences used are provided in the table below:

| | | SEQ | | SEQ |
|---|---|---|---|---|
| | Forward Primer | ID NO | Reverse Primer | ID NO |
| Coding | tctgcgaattcatgaccatccttttccttac | 21 | ttgatctcgagctatcttccccttttaatgg | 22 |
| Exon I | ctctctcgagtaaagcagtagccggctggt | 23 | ctctgaattctgtggctttgctgtcctgga | 24 |
| Exon II | ctctctcgaggctttggcaaagccatccac | 25 | ctctgaattccactcttctcacctggtgga | 26 |
| Exon IV | ctctctcgagacccactttcccattcaccg | 27 | ctctgaattccactcttctcacctggtgga | 26 |
| Exon VI | ctctctcgagccaatcgaagctcaaccgaa | 28 | ctctgaattccactcttctcacctggtgga | 26 |

Immunocytochemisty. Primary cultures of Bdnf^{NLuc/+} mouse neurons were plated onto PDL-coated 15 mm coverslips (Chemglass, Life Sciences) at the density of 8×10⁵ cells per well in 12-well plates. On DIV7, neurons were fixed with 4% paraformaldehyde supplemented with 120 mM sucrose in phosphate buffered saline (PBS) for 20 minutes and permeabilized with 0.25% Triton X-100 in PBS for 10 min at room temperature (RT). Neurons were then incubated with blocking buffer [0.1% Triton X-100, 10% bovine serum albumin (BSA) in PBS] for 1 hour at RT, and then incubated with primary antibody diluted in dilution buffer (0.1% Triton X-100, 1% BSA in PBS) for 2 hours at RT or overnight at 4° C. The following primary antibodies (Millipore) were used: NeuN (1:1,000; #ABN78), GFAP (1:500; #MAB360), MAP-2 (1:500; #MAB3418), PSD-95 (1:1,000; #MABN68), and synaptophysin (1:1,000; #MAB5258). After three washes in PBS, neurons were incubated with the appropriate secondary antibodies (1:500; Jackson ImmunoResearch) for 1 hour at RT, washed three times with PBS, and mounted to slides using DAPI Fluoromount-G medium (SouthernBiotech). Images were acquired using a Nikon C2+ confocal microscope.

Protein extraction and luciferase activity measurement. Cortices and striata of 10-week-old wild type (WT) or Bdnf^{NLuc/NLuc} mice were isolated and homogenized using RIPA lysis buffer supplemented with protease inhibitors (cOmplete™ Mini, Sigma). Protein extracts were obtained after lysates were centrifuged at 13,200 rpm for 20 min at 4° C. Protein concentration was determined with BCA assay kit (Thermo Fisher Scientific). Luciferase activity in protein extracts was detected using Nano-Glo Luciferase Assay System (Promega). Luminescence intensity was measured using microplate reader (FLUOstar Omega, BMG LABTECH) at 460 nm.

1. A rodent comprising a genetically modified endogenous Bdnf locus, wherein the genetically modified endogenous Bdnf locus encodes a BDNF-reporter fusion protein, wherein a nucleic acid sequence encoding the reporter is inserted in frame with the endogenous Bdnf coding sequence and within 1, 2, 3, 4, 5, 6, 7, 8, 9, or 10 codons of the stop codon of the Bdnf gene.

2. (canceled)

3. The rodent of claim 1, wherein the reporter comprises nano luciferase (NLuc).

4. The rodent of claim 3, wherein expression of the Bdfn-NLuc mRNA and/or the BDNF-NLuc fusion protein approximate the tissue distribution and physiological functions of wild type Bdnf mRNA and BDNF.

5. The rodent of claim 1, wherein the rodent is heterozygous for the genetically modified endogenous Bdnf locus.

6. The rodent of claim 1, wherein the rodent is homozygous for the genetically modified endogenous Bdnf locus.

7. (canceled)

8. The rodent of claim 1, wherein the rodent is a mouse.

9. The rodent of claim 8, wherein the coding sequence of the genetically modified endogenous Bdnf locus encodes a BDNF-NLuc fusion protein comprising the sequence set forth in SEQ ID NO: 8.

10. A method of making the rodent of claim 1, comprising:
(a) introducing into a rodent one-cell stage embryo:
(i) a donor template comprising an insert nucleic acid flanked by a 5′ homology arm that hybridizes to a 5′ target sequence at the endogenous Bdnf locus and a 3′ homology arm that hybridizes to a 3′ target sequence at the endogenous Bdnf locus, wherein the insert nucleic acid comprises the nucleic acid sequence encoding the reporter; and
(ii) a nuclease agent targeting a target sequence within the endogenous Bdnf locus,
wherein the genome is modified to comprise the genetically modified endogenous Bdnf locus; and
(b) implanting the modified rodent one-cell stage embryo into a surrogate mother to produce a genetically modified F0 generation rodent comprising the genetically modified endogenous Bdnf locus.

11. The method of claim 10, wherein the nuclease agent is a Cas9 protein and a guide RNA that targets a guide RNA target sequence within the endogenous Bdnf locus.

12. The method of claim 11, wherein step (a) further comprises introducing into the rodent one-cell stage embryo a second guide RNA that targets a second guide RNA target sequence within the endogenous Bdnf locus.

13. A method of assessing the activity of a molecule in modulating Bdnf expression, activity, and/or stability, comprising:
(a) isolating neuronal cells from the brains of embryos of the rodent of claim 1;
(b) contacting the isolated neuronal cells with the molecule in vitro; and
(c) assessing expression, activity, and/or stability of the reporter.

14. The method of claim 13, wherein the neuronal cells comprise cortical and/or hippocampal neurons.

15. The method of claim 13, wherein neuronal cells are isolated from about embryonic day 15.5 (E15.5) to about embryonic day 20.5 (E20.5) embryos.

16. The method of claim 15, wherein the neuronal cells are isolated at about embryonic day 18.5 (E18.5)

17. The method of claim 13, wherein the method further comprises:
(i) culturing the neuronal cells in Neurobasal medium containing about 2% fetal bovine serum (FBS), about 2% B27, about 2 mM GlutaMAX, about 50 U/mL penicillin/streptomycin (pen/strep), and about 16 μM 5-fluoro-2′-deoxyuridine (FUdR) for 3 to 12 days in vitro (DIV3-DIV12) at 37° C. and 5% CO₂ prior to step (c); and/or
(ii) plating the neuronal cells at a density of about 1.0×10⁴ cells per well in a 384-well plate or about 4.5×10⁴ cells per well in a 96-well plate.

18. The method of claim 13, wherein the neuronal cells are cultured for about 7 days in vitro (DIV7) prior to step (c).

19. The method of claim 13, wherein the reporter is NLuc, and wherein the assessing involves assessing expression, activity, and/or stability of luciferase activity in the neuronal cell population.

20. (canceled)

21. A neuronal cell population comprising a plurality of neuronal cells derived from the from the brains of embryos of the rodent of claim 1.

22. A method of assessing the activity of a BDNF-modulating molecule, comprising:
(a) administering the BDNF-modulating molecule to the rodent of claim 1; and
(b) assessing the activity of the reporter in the rodent.

23. The method of claim 22, wherein the reporter comprises NLuc and assessing the activity of the reporter in the rodent comprises bioluminescent imaging of the rodent, tissues from the rodent, or tissue extracts from the rodent.
